(19) United States
(12) Patent Application Publication
(10) Pub. No.: US 2018/0286505 A1
(43) Pub. Date: Oct. 4, 2018

(54) DRUG DOSAGE ALERTS AND RELATED METHODS AND MEDIA

(71) Applicants: CHILDREN'S HOSPITAL MEDICAL CENTER, Cincinnati, OH (US); UNIVERSITY OF CINCINNATI, Cincinnati, OH (US)

(72) Inventors: Eric KIRKENDALL, Cincinnati, OH (US); Judith Wehling DEXHEIMER, Wyoming, OH (US); Benjamin VAUGHAN, Cincinnati, OH (US)

(73) Assignees: CHILDREN'S HOSPITAL MEDICAL CENTER, Cincinnati, OH (US); UNIVERSITY OF CINCINNATI, Cincinnati, OH (US)

(21) Appl. No.: 15/944,829

(22) Filed: Apr. 4, 2018

Related U.S. Application Data
(60) Provisional application No. 62/481,243, filed on Apr. 4, 2017.

(51) Int. Cl. G16H 20/10; G16H 10/60

(52) U.S. Cl. CPC G16H 20/10; G08B 23/00; G16H 10/60

(57) ABSTRACT

Some embodiments of the invention include computer implemented methods for generating dosage alert values for a drug. Some embodiments include dosage alert values xₘᵢₙ and yₘᵢₙ for a drug, as determined by methods described herein. Other embodiments of the invention include non-transitory computer-readable media comprising computer program instructions executable by a computer processor to execute a method for generating dosage alert values for a drug. Still other embodiments of the invention include computer implemented methods for generating dosage alerts for a drug. Yet other embodiments of the invention include non-transitory computer-readable media comprising computer program instructions executable by a computer processor to execute a method for generating dosage alerts for a drug. Additional embodiments of the invention are also discussed herein.

### CROSS-REFERENCE TO RELATED APPLICATIONS

This application claims the benefit of U.S. Provisional Application No. 62/481,243, filed Apr. 4, 2017, entitled “Mathematical Model for Computer Assisted Modification of Medication Dosing Rules” which is herein incorporated by reference in its entirety.

### BACKGROUND

Computerized Provider Order Entry (CPOE) is a functionality of electronic health records (EHRs) and is a system where healthcare providers enter medication orders for use in patient care. Clinical decision support (CDS), which provides assistance in clinical decision-making, can be implemented within CPOE. Electronic medication rules (eRules) help the prescriber determine if a medication order is within reasonable and safe parameters based on factors such as the weight and age of a patient. If an order does not satisfy the conditions of the eRule, it will produce an alert to the prescriber.

Prescribers override CDS alerts for a variety of reasons. Since clinicians are familiar with traditional dosing patterns (often through textbooks and other sources) they will ignore alerts because they feel the system is giving them too many or that the alerts are inaccurate, clinically inappropriate, or simply not helpful. All these factors contribute to alert fatigue, which increases overrides of medication order-related CDS alerts. This raises safety concerns because clinicians are then more likely to ignore a correct alert (true positive) that may be lost amongst a sea of false positive alerts.

In pediatrics, for example, dosing eRules should be accurate because medication dosing can be complex due to weight-based factors (i.e., the weight of the patient) and can be prone to error. Unfortunately, the majority of the eRules in modern CDS systems are not tailored for pediatrics and orders in the pediatric space (e.g., orders entered into CPOE) can produce high numbers of alerts.

Improving the eRules to make the CDS more accurate could reduce alert burden (thereby improving alert and CDS salience) and dosing errors, thereby improving medication safety. Efforts have been made to change some of these rules. However, common vendor eRule databases contain hundreds of thousands of entries and minor changes in dosing parameters of one medication formulation may require the modification of dozens of other eRules due to the granularity of the databases. Currently there is no scalable and efficient solution to this problem.

Some of the embodiments of the invention address one or more of the deficiencies described above. For example, some embodiments of the invention include computer implemented methods for generating dosage alert values for a drug. Some embodiments include dosage alert values xₘᵢₙ and yₘᵢₙ for a drug, as determined by methods described herein. Other embodiments of the invention include non-transitory computer-readable media comprising computer program instructions executable by a computer processor to execute a method for generating dosage alert values for a drug. Still other embodiments of the invention include computer implemented methods for generating dosage alerts for a drug. Yet other embodiments of the invention include non-transitory computer-readable media comprising computer program instructions executable by a computer processor to execute a method for generating dosage alerts for a drug. Additional embodiments of the invention are also discussed herein.

### SUMMARY

Some embodiments of the invention include a computer implemented method for generating dosage alert values xₘᵢₙ and yₘᵢₙ for a drug, the method comprising minimizing I with respect to x, y, w₁, and w₂ for a dosage order database, where I=w₁*(A/N)+w₂*(|y−x|/L₀), xₘᵢₙ is the value of x when I is minimized and yₘᵢₙ is the value of y when I is minimized. In other embodiments, the xₘᵢₙ and yₘᵢₙ resulting from the minimization provide a percent alert rate improvement which is at least about 0%, at least about 0.1%, at least about 0.2%, at least about 0.3%, at least about 0.4%, at least about 0.5%, at least about 1%, at least about 1.5%, at least about 10%, at least about 25%, at least about 50%, at least about 75%, at least about 80%, at least about 85%, at least about 90%, at least about 95%, or at least about 99%. In certain embodiments, the xₘᵢₙ and yₘᵢₙ resulting from the minimization provide a number of dosage alerts saved per one hundred thousand dosage orders which is at least about 0, at least about 10, at least about 50, at least about 100, at least about 250, at least about 500, at least about 1000, at least about 5000, at least about 10,000, at least about 20,000, at least about 30,000, at least about 40,000, at least about 50,000, or at least about 90,000. Some embodiments include dosage alert values xₘᵢₙ and yₘᵢₙ, as determined by methods described herein.

Some embodiments of the invention include a non-transitory computer-readable medium comprising computer program instructions executable by a computer processor to execute a method for generating dosage alert values xₘᵢₙ and yₘᵢₙ for a drug using, for example, methods described herein.

Some embodiments of the invention include a computer implemented method for generating dosage alerts for a drug, the method comprising inputting a dosage request for a drug and generating a dosage alert if (a) the dosage request for the drug is less than xₘᵢₙ, (b) the dosage request for the drug is greater than yₘᵢₙ, or (c) both. In certain embodiments, xₘᵢₙ and yₘᵢₙ for the drug are generated using, for example, methods described herein. In other embodiments, the xₘᵢₙ and yₘᵢₙ resulting from a minimization method provide a percent alert rate improvement which is at least about 0%, at least about 0.1%, at least about 0.2%, at least about 0.3%, at least about 0.4%, at least about 0.5%, at least about 1%, at least about 1.5%, at least about 10%, at least about 25%, at least about 50%, at least about 75%, at least about 80%, at least about 85%, at least about 90%, at least about 95%, or at least about 99%. In certain embodiments, the xₘᵢₙ and yₘᵢₙ resulting from a minimization method provide a number of dosage alerts saved per one hundred thousand dosage orders which is at least about 0, at least about 10, at least about 50, at least about 100, at least about 250, at least about 500, at least about 1000, at least about 5000, at least about 10,000, at least about 20,000, at least about 30,000, at least about 40,000, at least about 50,000, or at least about 90,000.

Some embodiments of the invention include a non-transitory computer-readable medium comprising computer program instructions executable by a computer processor to execute a method for generating dosage alerts for a drug using, for example, methods described herein.

Other embodiments of the invention are also discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the description of specific embodiments presented herein.

FIG. 1: Illustrative flowcharts and pictures for some embodiments of the methods disclosed herein. (A) An illustrative embodiment of a method (100) used to determine dosage alert values (140) comprising minimizing I (130) using a dosage order database (110) and xₒᵣ and yₒᵣ (120). (B) An illustrative embodiment of a method (200) used to determine dosage alert values (260) comprising generating artificial dosages (220) for a historic dosage order database (210) to make a dosage order database (230) which is used along with xₒᵣ and yₒᵣ (240) to minimize I (250) and produce the dosage alert values (260). (C) An illustrative embodiment of a method (300) to test for a dosage alert (350) using dosage alert values (340), which dosage alert values are determined comprising minimizing I (330) using a dosage order database (310) and xₒᵣ and yₒᵣ (320). (D) An illustrative embodiment of a method (400) to test for a dosage alert (470) using dosage alert values (460), which dosage alert values (460) are determined comprising generating artificial dosages (420) from a historic dosage order database (410) to make a dosage order database (430) which together with xₒᵣ and yₒᵣ (440) is used to minimize I (450) and produce the dosage alert values (460). (E) An illustrative embodiment (500) showing some examples of the methods and plots to determine and evaluate dosage alert values comprising: retrieving a historic-based dosage database (501) to obtain a historic-based dosage database (502), generating artificial dosage orders (503) and obtaining a plot of the artificial dosage orders or the dosage order database (504), incorporating the artificial dosage order database or the dosage order database (505) into the model computation (506), calculating inferiority scores (507), plotting inferiority scores for various combinations of dosage alert values (508), minimizing dosage alert values for given weights (510), adding entries to a table (511), adjusting weight values (512) to provide adjusted weight values (513), and assessing dosage alert values (514).

FIG. 2: Artificial Dataset for (A) Acetaminophen and (B) Ursodiol 0-12 years. The large histograms show two examples of the distribution of the artificial dataset doses. The histograms in the insets show the less frequent doses.

FIG. 3: Surface Plots of Dosing Rule Interval Limits; Grid output from the optimization algorithm. Each dosing rule limits considered between 0 and 20 mg/kg was assigned an inferiority score. The lowest inferiority score denotes the optimal dosing range as defined by a priori criteria. In both A and B the lowest inferiority score returned was 10-15 mg/kg in this instance of weight values, thus the optimal dosing interval for both Acetaminophen and Ursodiol (0-12 years) is 10-15 mg/kg.

FIG. 4: Visual Representation of Rule % Improvement vs Actual eRule. ‘Actual eRule’ (e.g., xₒᵣ and yₒᵣ) label is directly beneath the bar representing the parameters or the actual dosing rule in clinical use. Other bars represent the comparison to different algorithmically-derived dosing rule parameter choices. (A) Ibuprofen shows about 3% improvement in alert savings if the dosing rule is 5-15 mg/kg versus the actual eRule of 4-11 mg/kg. (B) Ursodiol shows significant improvement in alert reduction if the rule range is increased.

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include computer implemented methods for generating dosage alert values for a drug. Some embodiments include dosage alert values xₘᵢₙ and yₘᵢₙ for a drug, as determined by methods described herein. Other embodiments of the invention include non-transitory computer-readable media comprising computer program instructions executable by a computer processor to execute a method for generating dosage alert values for a drug. Still other embodiments of the invention include computer implemented methods for generating dosage alerts for a drug. Yet other embodiments of the invention include non-transitory computer-readable media comprising computer program instructions executable by a computer processor to execute a method for generating dosage alerts for a drug. Additional embodiments of the invention are also discussed herein.

Some embodiments of the invention include methods for determining if a dosage alert should be produced. A dosage alert can be in any form that alerts the user, including but not limited to one or more sounds, one or more lights, one or more text messages, one or more messages (e.g., text, text bolding, text highlight color changes, color changes) on a screen (e.g., a phone screen, a tablet screen, a monitor screen etc.), one or more spoken messages, one or more tactile sensations (e.g., a vibration), one or more taste sensations, one or more smell sensations, or a combination thereof. Any suitable user can receive the one or more dosage alerts, including but not limited to any prescriber (e.g., doctor, medical doctor, doctor of osteopathic medicine, physician's assistant, nurse, registered nurse, nurse practitioner, paramedic, or emergency medical technician) or person who can assist the prescriber (e.g., any nurse assistant, any doctor assistant, doctor of osteopathic medicine, physician's assistant, nurse, registered nurse, nurse practitioner, paramedic, or emergency medical technician).

The term “drug” as used herein means any medication or pharmaceutical that can be obtained over-the-counter, that may require a prescription, that may not require a prescription, or that can be prescribed. In some embodiments, the drug can be any drug listed in <<https://www.drugs.com/drug_information.html>>. In other embodiments, the drug can be abatacept, abiraterone, acetaminophen, acetaminophen/hydrocodone, adalimumab, adderall, albuterol sulfate, alprazolam, amitriptyline, amlodipine, amoxicillin, amoxicillin-pot clavulanate, amphetamine mixed salts, analgesics-narcotic, analgesics-nonnarcotic, antianxiety agents, antiasthmatic, anticonvulsant, antidepressants, antihistamines, antiinfectives, anti-rheumatic, aripiprazole, atazanavir, ativan, atorvastatin, azithromycin, bevacizumab, budesonide, budesonide/formoterol, buprenorphine, capecitabine, celecoxib, cephalosporins, chlorothiazide, ciclosporin ophthalmic emulsion, cinacalcet, ciprofloxacin, citalopram, clindamycin, clindamycin HCl, clindamycin palmitate HCl, clonazepam, codeine, corticosteroids, cyclobenzaprine, cymbalta, dabigatran, darbepoetin alfa, darunavir, denosumab, dexlansoprazole, diazepam, diphenhydramine HCl, doxycycline, duloxetine, elvitegravir/cobicistat/emtricitabine/tenofovir, emtricitabine/rilpivirine/tenofovir disoproxil fumarate, emtricitabine/tenofovir/efavirenz, enoxaparin, epoetin alfa, esomeprazole, eszopiclone, etanercept, everolimus, ezetimibe, ezetimibe/simvastatin, fenofibrate, filgrastim, fingolimod, fluticasone propionate, fluticasone propionate/salmeterol, fluticasone/salmeterol, furosemide, gabapentin, gastrointestinal agents, glatiramer, hydrochlorothiazide, hydromorphone HCl, hypnotics, ibuprofen, imatinib, infliximab, insulin aspart, insulin detemir, insulin glargine, insulin lispro, interferon beta 1b, ipratropium bromide/salbutamol, laxatives, ledipasvir/sofosbuvir, lenalidomide, levetiracetam, levothyroxine, lexapro, lidocaine, liraglutide, lisdexamfetamine, lisinopril, local anesthetics-parenteral, loratadine, lorazepam, losartan, lyrica, melatonin, meloxicam, memantine, metformin, methadone HCl, methylphenidate, metoprolol, metoprolol, mometasone, naproxen, olmesartan, olmesartan/hydrochlorothiazide, omalizumab, omega-3 fatty acid ethyl esters, omeprazole, ondansetron HCl, ophthalmic, oxycodone, palivizumab, pantoprazole, pemetrexed, penicillin v potassium, penicillins, pneumococcal conjugate vaccine, polymyxin b-trimethoprim, prednisolone sodium phosphate, prednisone, pregabalin, quetiapine, rabeprazole, raloxifene, raltegravir, ranibizumab, ranitidine HCl, rituximab, rivaroxaban, rocuronium bromide, rosuvastatin, salbutamol, sevelamer, sildenafil, sitagliptin, sitagliptin/metformin, sofosbuvir, solifenacin, stimulants, sulfamethoxazole-trimethoprim, tacrolimus, tadalafil, telaprevir, tenofovir/emtricitabine, testosterone gel, tiotropium bromide, tramadol, trastuzumab, trazodone, ulcer drugs, ursodiol, ursodiol, ustekinumab, valproate, valsartan, viagra, wellbutrin, xanax, zoloft, or Zostavax.

In certain embodiments, the drug can be acetaminophen, albuterol sulfate, amoxicillin, amoxicillin-pot clavulanate, chlorothiazide, clindamycin HCl, clindamycin palmitate HCl, diazepam, diphenhydramine HCl, epoetin alfa, furosemide, hydromorphone HCl, levetiracetam, lorazepam, melatonin, methadone HCl, ondansetron HCl, penicillin v potassium, polymyxin b-trimethoprim, prednisolone sodium phosphate, ranitidine HCl, rocuronium bromide, sulfamethoxazole-trimethoprim, tacrolimus, or ursodiol. In other embodiments, the drug does not include one or more of acetaminophen, albuterol sulfate, amoxicillin, amoxicillin-pot clavulanate, chlorothiazide, clindamycin HCl, clindamycin palmitate HCl, diazepam, diphenhydramine HCl, epoetin alfa, furosemide, hydromorphone HCl, levetiracetam, lorazepam, melatonin, methadone HCl, ondansetron HCl, penicillin v potassium, polymyxin b-trimethoprim, prednisolone sodium phosphate, ranitidine HCl, rocuronium bromide, sulfamethoxazole-trimethoprim, tacrolimus, or ursodiol. In certain embodiments, the drug does not include any of acetaminophen, albuterol sulfate, amoxicillin, amoxicillin-pot clavulanate, chlorothiazide, clindamycin HCl, clindamycin palmitate HCl, diazepam, diphenhydramine HCl, epoetin alfa, furosemide, hydromorphone HCl, levetiracetam, lorazepam, melatonin, methadone HCl, ondansetron HCl, penicillin v potassium, polymyxin b-trimethoprim, prednisolone sodium phosphate, ranitidine HCl, rocuronium bromide, sulfamethoxazole-trimethoprim, tacrolimus, and ursodiol.

In other embodiments, the drug can be acetaminophen, amoxicillin, diphenhydramine, ibuprofen, ursodiol for ages 0-12, or ursodiol for ages 12-99.

In some embodiments, the drug can be dosed heterogeneously. In other embodiments, the drug is not dosed heterogeneously. For example, in some clinical environments, one or more of acetaminophen, diphenhydramine, or ibuprofen are dosed heterogeneously. In other clinical environments, one or more of acetaminophen, diphenhydramine, or ibuprofen are not dosed heterogeneously. In some embodiments, heterogeneous dosing refers to the quality or state of being diverse in the character or content of the dosages. In other embodiments, heterogeneous dosing refers to dosing that ranges widely (e.g., dosages that change per prescriber, context, etc.). For example, acetaminophen can, in some instances, be dosed not heterogeneously in that it can be dosed at 15 mg/kg—e.g., so many weight-based dosages in a dosage order database (e.g., historic dosage order database) could be 15 mg/kg. In another example, ursodiol can be dosed heterogeneously in that it can be dosed depending on one or more factors, and the dosage data in a dosage order database (e.g., historic dosage order database) reflect those variances (e.g., dosages can be 2 mg/kg, 5 mg/kg, 8 mg/kg, etc.).

In certain embodiments, a drug can be subdivided (i.e., treated as a separate drug for the purposes of the method) using any other suitable factor including but not limited to one or more of: by age (e.g., subdividing ursodiol by age, such as, ursodiol for ages 0-12 and ursodiol for ages 12-99), by weight (e.g., the dosage can increase if the weight of the person increases), by disease, by kidney status, by liver status, allergies (e.g., a person allergic to one drug may require more limited dosages of another drug), by disorder or condition (e.g., the dosage of an anti-cancer drug when used to treat a systemic cancer might be differently dosed when compared to that same drug used to treat cancer locally, such as a non-metastatic tumor), by severity or stage of a disease, disorder, or condition (e.g., treatment of a stage I cancer might require a different dosage as compared to treatment of stage IV cancer), when used in combination with another drug (e.g., acetaminophen when used in combination ibuprofen for high fevers or amoxicillin when used in combination with clavulanate for certain infections), by genetic disposition (e.g., ability to metabolize a certain drug faster or slower), by bodily condition (e.g., if the animal is under a fast or famine condition, or vitamin/nutrient depravation or overdose, or is addicted to or having an overdose of a drug), by environmental condition (e.g., if the animal has been or will be exposed to certain environments or environmental conditions) or by formulation (e.g., suspension, reconstituted suspension, tablet, chewable tablet, solution, capsule, gel, tablet dispersible, elixir, syrup, liquid). In some embodiments, drugs administered topically are excluded. The definition of “drug” as used herein can, in some embodiments, be sub-divided according to one or more of the above categories. For example, the “drug” of ursodiol for ages 0-12 can be a separate and distinct drug compared to the “drug” of ursodiol for ages 12-99. As another example, formulations can each be treated separately as a distinct drug, or several formulations can be grouped together as a single drug.

In some embodiments, a computer implemented method can be used for generating dosage alert values xₘᵢₙ and yₘᵢₙ for a drug. In certain embodiments, the method comprises minimizing I with respect to x, y, w₁, and w₂ for a dosage order database. I (the measure of inferiority) is equal to w₁*(A/N)+w₂*(|y−x|/L₀). “*” means multiplication, “/” means division, and “|y−x|” means the absolute value of y minus x. The dosage order database comprises dosage orders for the drug. A dosage in the dosage order database that is less than x generates an alert and a dosage in the dosage order database that is greater than y generates an alert. A is the number of alerts generated by the dosage orders in the dosage order database with given values of x and y. N is the total number of orders in the dosage order database for the drug. w₁ and w₂ are weights (e.g., arbitrary, one or both are allowed to be varied, one or both are fixed, or one is set to a value relative to the other). L₀ is yₒᵣ−xₒᵣ, where xₒᵣ is an original dosage alert value for the drug (where a dosage in the dosage order database below xₒᵣ will generate an alert) and yₒᵣ is an original dosage alert value for the drug (where a dosage in the dosage order database above yₒᵣ will generate an alert). xₘᵢₙ is the value of x when I is minimized, and yₘᵢₙ is the value of y when I is minimized.

In some embodiments, xₒᵣ, yₒᵣ, or both can come from any suitable source. In some embodiments, xₒᵣ, yₒᵣ, or both can come from electronic medication rules, an enterprise electronic health records (EHR) system used by an institution (e.g., EHR, Verona Wis.), a supplement to an EHR (e.g., Medi-Span (Wolters Kluwer Health, Philadelphia, Pa.), custom dosing rules (e.g., created by an institution, a pharmacy, a doctor, a pharmacist, other medical persons, a software engineer/designer, or combinations thereof), or a combination thereof. In some embodiments, xₒᵣ, yₒᵣ, or both can result from customization of a prior xₒᵣ, yₒᵣ, or both. In some embodiments, xₒᵣ, yₒᵣ, or both does not result from customization of a prior xₒᵣ, yₒᵣ, or both. In certain embodiments, xₒᵣ, yₒᵣ, or both can come from prior calculated xₘᵢₙ, yₘᵢₙ, or both, but which can then be applied to an I minimization using a different dosage order database (e.g., a dosage order database used in the minimization of I to yield xₘᵢₙ and yₘᵢₙ, but which dosage order database has been updated or includes more recent dosage order entries which may or may not have used the xₘᵢₙ and yₘᵢₙ as dosage alert values during that more recent dosage order entries).

In certain embodiments, one of the terms (i.e., w₁*(A/N) or w₂*(|y−x|/L₀)) is scaled (e.g., by the choice of w₁ or w₂) to be less than one and the other term is scaled to be close to having a value of one (e.g., about 0.8, about 0.85, about 0.9, about 0.92, about 0.95, about 0.97, about 0.98, about 0.99, about 1, about 1.01, about 1.02, about 1.03, about 1.04, about 1.05, about 1.08, about 1.1, about 1.15, or about 1.2). In other embodiments, w₁ is greater than w₂. In some embodiments, w₁ is less than w₂. In other embodiments, w₂ is fixed at 1. In yet other embodiments, w₁ is fixed at 1. In still other embodiments, w₁ is greater than w₂ and w₂ is fixed at 1.

In some embodiments, the dosage order database can be taken from or adapted from any suitable database. The dosage order database can be obtained or retrieved using any suitable method. In certain embodiments, the dosage order database can include dosage orders from a database that were given to a patient, dosage orders canceled (e.g., by a prescriber), dosage orders removed (e.g., by a prescriber), dosage orders that received an alert, dosage orders that did not receive an alert, or combinations thereof. For example, in certain embodiments, the dosage order database can be a historic-based database (e.g., a historic-based dosage order database). In some embodiments, the historic-based database can be taken from one or more historic dosage order databases, using all of the doses in one or more of the historic dosage order databases, some of the doses in the one or more historic databases, or a combination thereof. In certain embodiments, a dosage order database can be a historic dosage order database from an institution, from a combination of selected institutions, from a combination of institutions in a geographic region (e.g., metropolitan area, regional area (e.g., a tri-state area or multi-city area), a single state, groups of states (e.g., eastern states, western states, or midwestern states), a single country, borderland areas/regions of two or more countries, or multi-country regions), from a combination of selected institutions that treat the same or similar type or class of patients (e.g., children's hospitals or veteran's hospitals), from a single or combination of institutions that specialize or have expertise for certain conditions, or combinations thereof. In other embodiments, a dosage order database can be from the one or more historic dosage order databases and dosages can be selected from one or more of the historic dosage order databases based on one or more of age group, disease or disorder, genetic predisposition, environmental predisposition, allergies, injury type, the time frame of when the drug was ordered (months, years, decades, centuries, or based upon time relation when certain events occurred, such as disease outbreaks or environmental events), or combinations thereof.

In some embodiments, the dosage order database can be generated by adding any amount of any suitable data to a historic-based database. In certain embodiments, a historic-based database may not be sufficient to produce adequate (e.g., sufficiently defined) values of one or more of xₘᵢₙ, yₘᵢₙ, w₁, and w₂, such as but not limited to historic-based databases for drugs that are infrequently ordered. In other embodiments, the dosage order database can be generated from one or more historic-based dosage order databases by incorporating artificial dosage data (e.g., by incorporating unseen doses that could appear in the future). In some embodiments, artificial dosage data (e.g., unseen doses that could appear in the future) can be calculated using any suitable method including but not limited to a fequentist statistical model (e.g., a maximum likelihood estimation (MLE), a Good-Turing frequency estimation (GTE), or any suitable extension or variation thereof) or a Baysian model. In some embodiments, the artificial dosage data assumes that future dosage ordering patterns are similar to past dosage ordering patterns. In some instances, the frequentist statistical model or Baysian model can incorporate a cumulative distribution function to generate artificial dosage data. Some or all of the artificial dosage data can, in certain instances, be combined with some or all of the historic-based databases to generate the dosage order database.

In some embodiments, the dosage order database can be an updated (e.g., corrected by removing or fixing prior errors, such as data entry errors) dosage order database. In other embodiments, the dosage order database can include more recent dosage order entries, such as but not limited to entries which occurred after implementation of an xₘᵢₙ and yₘᵢₙ as dosage alert values (i.e., xₘᵢₙ and yₘᵢₙ obtained from minimization of I using the dosage order database prior to the inclusion of more recent dosage order entries).

In some embodiments, the dosage order database can comprise about 10,000 dosage orders, about 50,000 dosage orders, about 100,000 dosage orders, about 200,000 dosage orders, about 300,000 dosage orders, about 400,000 dosage orders, about 500,000 dosage orders, about 1,000,000 dosage orders, about 2,000,000 dosage orders, about 5,000,000 dosage orders, about 10,000,000 dosage orders, at least about 10,000 dosage orders, at least about 50,000 dosage orders, at least about 100,000 dosage orders, at least about 200,000 dosage orders, at least about 300,000 dosage orders, at least about 400,000 dosage orders, at least about 500,000 dosage orders, at least about 1,000,000 dosage orders, at least about 2,000,000 dosage orders, at least about 5,000,000 dosage orders, or at least about 10,000,000 dosage orders.

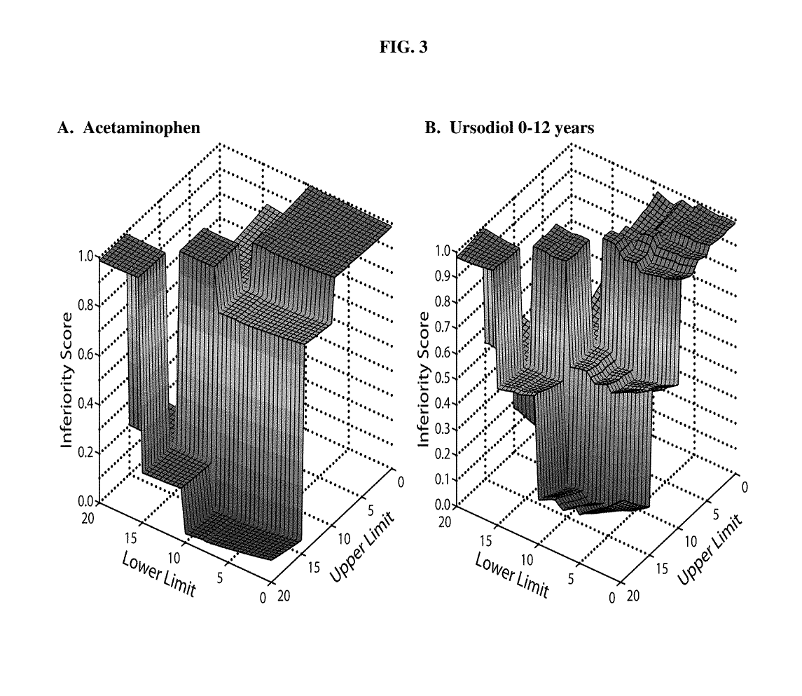

In certain embodiments, the step of minimizing I (i.e., the measure of inferiority) with respect to x, y, w₁, and w₂, can use any suitable minimization algorithm, including but not limited to brute force, Powell's conjugate direction method, or the Nelder-Mead method. In some instances, for the minimization step, one of the terms (i.e., w₁*(A/N) or w₂*(|y−x|/L₀)) can be scaled (e.g., by the choice of w₁ or w₂) to be less than one and the other term can be scaled to be close to having a value of one (e.g., about 0.8, about 0.85, about 0.9, about 0.92, about 0.95, about 0.97, about 0.98, about 0.99, about 1, about 1.01, about 1.02, about 1.03, about 1.04, about 1.05, about 1.08, about 1.1, about 1.15, or about 1.2). In other embodiments, for the minimization step, w₁>w₂. In some embodiments, for the minimization step, w₁<w₂. In other embodiments, for the minimization step, w₂ can be fixed at 1. In yet other embodiments, for the minimization step, w₁ can be fixed at 1. In still other embodiments, for the minimization step, w₁>w₂ and w₂ is fixed at 1. In some instances (e.g., for visualization purposes), I is minimized by varying w₁ and w₂ (e.g., using any manner, choices, or relationships of w₁ and w₂, as disclosed herein) for fixed, pre-selected values x and y; in certain embodiments, I can be plotted as a function of the pre-selected values of x and y (e.g., see FIG. 3). These plots can be used for any suitable purpose, including but not limited to minimizing I by visually examining these plots (e.g., for a fixed w₁ and w₂) or using the plots to better understand or evaluate the nature of the choice of x and y on the inferiority score.

In some embodiments, the dosage alert values xₘᵢₙ and yₘᵢₙ resulting from the minimization can be assessed using any suitable method (e.g., comparing to other dosage alert values, comparing to dosage alert values of xₒᵣ and yₒᵣ, or evaluating the suitability of the dosage alert values as compared to other dosage alert values such as xₒᵣ and yₒᵣ). In certain embodiments, the dosage alert values xₘᵢₙ and yₘᵢₙ resulting from the minimization can be assessed using the alert rate or number of dosage alerts saved per one hundred thousand dosage orders.

In some embodiments, the alert rate can be calculated; the alert rate is defined as the percent of dosage orders in a dosage order database that generate an alert for a given rule (i.e., for a given x and y). Thus, the “percent alert rate improvement” of the alert rate of calculated dosage alert values compared to the alert rate of the original dosage alert values (i.e., (original alert rate-calculated alert rate)/original alert rate) can be a measure of an improvement of other calculated dosage alert values compared to original dosage alert values (e.g., for a given dosage alert database). In certain embodiments, the other calculated dosage alert values are xₘᵢₙ and yₘᵢₙ resulting from an I minimization. In some embodiments, the percent alert rate improvement can be positive or negative and can be about 0%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, at least about 0%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.5%, at least about 2%, at least about 2.5%, at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the number of dosage alerts saved per one hundred thousand dosage orders (e.g., for a given dosage alert database) can be calculated by subtracting the number of dosage alerts per 100,000 dosage orders using the original dosage alert values, from the number of dosage alerts per 100,000 dosage orders using other calculated dosage alert values. In certain embodiments, the other calculated dosage alert values are xₘᵢₙ and yₘᵢₙ resulting from an I minimization. The number of dosage alerts saved per one hundred thousand dosage orders can be a measure of an improvement of calculated dosage alert values compared to original dosage alert values. In some embodiments, the number of dosage alerts saved per one hundred thousand dosage orders can be positive or negative, about 0, about 1, about 2, about 3, about 4, about 5, about 10, about 20, about 50, about 100, about 250, about 500, about 750, about 1000, about 2000, about 5000, about 10,000, about 20,000, about 30,000, about 40,000, about 50,000, about 60,000, about 70,000, about 80,000, about 90,000, about 100,000, at least about 0, at least about 1, at least about 2, at least about 3, at least about 4, at least about 5, at least about 10, at least about 20, at least about 50, at least about 100, at least about 250, at least about 500, at least about 750, at least about 1000, at least about 2000, at least about 5000, at least about 10,000, at least about 20,000, at least about 30,000, at least about 40,000, at least about 50,000, at least about 60,000, at least about 70,000, at least about 80,000, or at least about 90,000.

Some embodiments of the invention include a non-transitory computer-readable medium comprising computer program instructions executable by a computer processor to execute a method for generating dosage alert values xₘᵢₙ and yₘᵢₙ for a drug according to any suitable method, including those disclosed herein (e.g., those disclosed above). In some embodiments, the drug used can be any suitable drug such as those disclosed herein (e.g., those disclosed above). In certain embodiments, the drug is acetaminophen, amoxicillin, diphenhydramine, ibuprofen, or ursodiol for ages 0-12. In other embodiments, the xₘᵢₙ and yₘᵢₙ resulting from the minimization provide a percent alert rate improvement that can be any suitable value such as those disclosed herein (e.g., those disclosed above). In some embodiments, the xₘᵢₙ and yₘᵢₙ resulting from the minimization provide a percent alert rate improvement that can be at least about 0%, at least about 0.1%, at least about 0.2%, at least about 0.3%, at least about 0.4%, at least about 0.5%, at least about 1%, at least about 1.5%, at least about 10%, at least about 25%, at least about 50%, at least about 75%, at least about 80%, at least about 85%, at least about 90%, at least about 95%, or at least about 99%. In other embodiments, the xₘᵢₙ and yₘᵢₙ resulting from the minimization can provide a number of dosage alerts saved per one hundred thousand dosage orders that can be any suitable value such as those disclosed herein (e.g., those disclosed above). In other embodiments, the xₘᵢₙ and yₘᵢₙ resulting from the minimization can provide a number of dosage alerts saved per one hundred thousand dosage orders that can be at least about 0, at least about 10, at least about 50, at least about 100, at least about 250, at least about 500, at least about 1000, at least about 5000, at least about 10,000, at least about 20,000, at least about 30,000, at least about 40,000, at least about 50,000, or at least about 90,000.

Some embodiments of the invention include a computer implemented method for generating dosage alerts for a drug using any suitable method. In other embodiments, the method for generating one or more dosage alerts for a drug can comprise inputting a dosage request for a drug and generating a dosage alert if (a) the dosage request for the drug is less than xₘᵢₙ, (b) the dosage request for the drug is greater than yₘᵢₙ, or (c) both. The values of xₘᵢₙ and yₘᵢₙ for the drug can be generated using any suitable method including those described herein (e.g., those described above). In yet other embodiments, the inputting can occur using any suitable device (e.g., those described herein) including but not limited to a keyboard, a pointing device, a microphone, a touch screen, or a combination thereof. In still other embodiments, the the dosage request can be performed by any suitable person or machine including but not limited to a prescriber or a person who assists a prescriber. In some embodiments, the dosage alert can be any suitable alert (e.g., those described herein) including but not limited to a sound, a light, a text message, a message on a screen, a spoken message, a tactile sensation, or a combination thereof. In yet other embodiments, the dosage alert can be received by any suitable person or machine including but not limited to a prescriber or a person who assists a prescriber. In still other embodiments, the drug can be any suitable drug including those described herein (e.g., those described above). In some embodiments, the drug can be acetaminophen, amoxicillin, diphenhydramine, ibuprofen, or ursodiol for ages 0-12. In other embodiments, the xₘᵢₙ and yₘᵢₙ resulting from an I minimization provide a percent alert rate improvement that can be any suitable value such as those disclosed herein (e.g., those disclosed above). In some embodiments, the xₘᵢₙ and yₘᵢₙ resulting from the minimization provide a percent alert rate improvement that can be at least about 0%, at least about 0.1%, at least about 0.2%, at least about 0.3%, at least about 0.4%, at least about 0.5%, at least about 1%, at least about 1.5%, at least about 10%, at least about 25%, at least about 50%, at least about 75%, at least about 80%, at least about 85%, at least about 90%, at least about 95%, or at least about 99%. In other embodiments, the xₘᵢₙ and yₘᵢₙ resulting from an I minimization can provide a number of dosage alerts saved per one hundred thousand dosage orders that can be any suitable value such as those disclosed herein (e.g., those disclosed above). In other embodiments, the xₘᵢₙ and yₘᵢₙ resulting from an I minimization can provide a number of dosage alerts saved per one hundred thousand dosage orders that can be at least about 0, at least about 10, at least about 50, at least about 100, at least about 250, at least about 500, at least about 1000, at least about 5000, at least about 10,000, at least about 20,000, at least about 30,000, at least about 40,000, at least about 50,000, or at least about 90,000.

Some embodiments of the invention include a non-transitory computer-readable medium comprising computer program instructions executable by a computer processor to execute a method for generating dosage alerts for a drug according to any suitable method including those described herein. In other embodiments, the method for generating one or more dosage alerts for a drug can comprise inputting a dosage request for a drug and generating a dosage alert if (a) the dosage request for the drug is less than xₘᵢₙ, (b) the dosage request for the drug is greater than yₘᵢₙ, or (c) both. The values of xₘᵢₙ and yₘᵢₙ for the drug can be generated using any suitable method including those described herein (e.g., those described above). In yet other embodiments, the inputting can occur using any suitable device (e.g., those described herein) including but not limited to a keyboard, a pointing device, a microphone, a touch screen, or a combination thereof. In still other embodiments, the the dosage request can be performed by any suitable person or machine including but not limited to a prescriber or a person who assists a prescriber. In some embodiments, the dosage alert can be any suitable alert (e.g., those described herein) including but not limited to a sound, a light, a text message, a message on a screen, a spoken message, a tactile sensation, or a combination thereof. In yet other embodiments, the dosage alert can be received by any suitable person or machine including but not limited to a prescriber or a person who assists a prescriber. In still other embodiments, the drug can be any suitable drug including those described herein (e.g., those described above). In some embodiments, the drug can be acetaminophen, amoxicillin, diphenhydramine, ibuprofen, or ursodiol for ages 0-12. In other embodiments, the xₘᵢₙ and yₘᵢₙ resulting from an I minimization provide a percent alert rate improvement that can be any suitable value such as those disclosed herein (e.g., those disclosed above). In some embodiments, the xₘᵢₙ and yₘᵢₙ resulting from an I minimization provide a percent alert rate improvement that can be at least about 0%, at least about 0.1%, at least about 0.2%, at least about 0.3%, at least about 0.4%, at least about 0.5%, at least about 1%, at least about 1.5%, at least about 10%, at least about 25%, at least about 50%, at least about 75%, at least about 80%, at least about 85%, at least about 90%, at least about 95%, or at least about 99%. In other embodiments, the xₘᵢₙ and yₘᵢₙ resulting from an I minimization can provide a number of dosage alerts saved per one hundred thousand dosage orders that can be any suitable value such as those disclosed herein (e.g., those disclosed above). In other embodiments, the xₘᵢₙ and yₘᵢₙ resulting from an I minimization can provide a number of dosage alerts saved per one hundred thousand dosage orders that can be at least about 0, at least about 10, at least about 50, at least about 100, at least about 250, at least about 500, at least about 1000, at least about 5000, at least about 10,000, at least about 20,000, at least about 30,000, at least about 40,000, at least about 50,000, or at least about 90,000.

Patient privacy and patient confidentiality have been ongoing considerations in medical environments for many years. Thus, medical facility and pharmaceutical personnel may provide permission forms for patient review and signature before the patient's information (e.g., dosing information or medical conditions that could influence dosing) is entered into an electronic information system, to ensure that a patient is informed of potential risks of electronically stored personal/private information such as a medical history or other personal identifying information. Further, authentication techniques may be included in order for medical facility and/or pharmaceutical facility personnel to enter or otherwise access patient information in the system. For example, a user identifier and password may be requested for any type of access to patient information. As another example, an authorized fingerprint or audio identification (e.g., via voice recognition) may be requested for the access. Additionally, access to networked elements of the system may be provided via secured connections (or hardwired connections), and firewalls may be provided to minimize risk of potential hacking into the system.

Further, medical and/or pharmaceutical facility personnel may provide permission forms for facility employees for review and signature before the employees' information is entered into an electronic medical information system, to ensure that employees are informed of potential risks of electronically stored personal/private information such as a medical history or other personal identifying information.

The presently-disclosed subject matter is further illustrated by the following specific but non-limiting examples. The following examples may include compilations of data that are representative of data gathered at various times during the course of development and experimentation related to the present invention.

### EXAMPLES

### Materials and Methods

Setting

Cincinnati Children's Hospital Medical Center is a level 1 trauma center with 628 licensed beds. It has 1.2 million patient encounters annually, including 30,000 admissions, 33,000 surgeries, 900,000 ambulatory encounters, and 125,000 emergency department visits. There are approximately 200,000 medication orders a month, which generate 75,000 dosing alerts. The institution implemented an enterprise EHR (EpicCare®, Verona, Wis., USA) in 2007. The EHR is configured to use a combination of the Medi-Span (Wolters Kluwer Health, Philadelphia, Pa.) drug dosing decision support rules and supplemented with custom dosing rules created and maintained by the pharmacy.

Dosing Rules Description

There are two sets of dosing rules used in this study: actual dosing rules, and the model-generated dosing rules. The actual dosing rules are the electronic rules (eRules) that are used in the study site's production CPOE system rules engine and clinical care. The actual dosing rules (xₒᵣ and yₒᵣ, as discussed herein) were purchased from a third-party vendor. All the dosing parameters for the candidate medications selected in this study were already customized and overrode the vendor's default rule parameters. The model-generated dosing rules (xₘᵢₙ and yₘᵢₙ, as discussed herein) are the dosing rules output by the mathematical model created to optimize the rule parameters. The two sets of dosing rules represent different CPOE configurations for weight-based dosing clinical decision support. The theoretical performances of these sets of dosing rules were compared to demonstrate the viability and feasibility of the model-generated dosing rules.

Clinical Dataset Used to Derive the Model

Historical medication order data from 2011 to 2015 was retrieved from a previously described decision support analytic data warehouse. Five medications were selected to use in developing the model: Acetaminophen, Ibuprofen, Diphenhydramine, Amoxicillin, and Ursodiol. These medications were selected based on criteria that included frequency of ordering and frequency of alerting. Ursodiol was selected because it was previously known to have a poor match between the formal dosing eRules and how it is ordered in practice. We aggregated different formulations for the selected medications based on ordering behavior (Table 1). For example, in our dataset Ibuprofen has formulations of suspension, tablets, chewable tablets, solution, and capsule. These were grouped together for analysis since those formulations are typically prescribed with the same dosing guidelines. The data was filtered for orders placed using weight-based dosing, e.g., in mg/kg. Orders placed using absolute dosing, e.g., in milligrams, were excluded since the model calculated weight-based dosing parameters only. The data includes all medication dosing orders that were attempted as well as orders canceled and removed by prescribers after they attempted to sign the orders and received an alert.

TABLE 1
Medication Data Counts, Formulation Groupings, and
Dosing Rule Parameters (Actual eRule)

| Medication | Order Count |
|---|---|
| Acetaminophen | 376794 |
| Ibuprofen | 163225 |
| Diphenhydramine | 42884 |
| Amoxicillin | 61323 |
| Ursodiol 0-12 | 1191 |
| Ursodiol 12-99 | 39 |
| Total | 645456 |

Medication Formulations
Diphenhydramine

| Actual eRule | 0.1-1.5 mg/kg |
|---|---|
| Liquid | Cap |
| Elixir | Solution |
| Chew Tab | Tab |

Medication Formulations
Ursodiol

| | |
|---|---|
| Actual eRule 0-12 | 10-15 mg/kg |
| Actual eRule 12-99 | 2-5 mg/kg |
| Cap | Suspension |

Medication Formulations
Acetaminophen

| Actual eRule | 5-15 mg/kg |
|---|---|
| Suspension | Gel |
| TABLET | Chew Tab |
| DISPERSIBLE | |
| Elixer | Cap |
| Syrup | Solution |
| Liquid | Tab |

Medication Formulations
Ibuprofen

| Actual eRule | 4-11 mg/kg |
|---|---|
| Suspension | Solution |
| Tab | Cap |
| Chew Tab | |

Medication Formulations
Amoxicillin

| Actual eRule | 8-45 mg/kg |
|---|---|
| Tab | Cap |
| Chew Tab | Recon Susp |

*Ursodiol has a different eRule for different age groups: the split was based on the construct of the current eRules.

Generation of the Artificial Dataset

The mathematical model and optimization algorithms require sufficient dosing data to provide meaningful dosing rules. For frequently ordered medications, historical data could be used to generate dosing rule but for infrequently ordered medications, historical data may not be sufficient to produce effective dosing rules. Additionally, historical data would not contain unseen doses that could appear in the future.

To generate a sufficiently large set of dose orders and to incorporate possibly unseen does, we created a frequentist statistical model based on historical ordering behavior to generate artificial data to predict future ordering patterns for the model, assuming future ordering patterns are similar to past ordering patterns. Other approaches to generate meaningful artificial data such as Baysian models can be used in lieu of this approach since the mathematical model and optimization algorithm are indifferent to the origin of the data.

For large historical dosing datasets from commonly prescribed medications such as Acetaminophen or Ibuprofen, the probability of a specific dose can be estimated using the maximum likelihood estimate (MLE) where the probability is the frequency of the specific dose divided by the total number of orders. A possible limitation in this approach is that doses that do not appear in the historical dataset have a zero probability of occurring in the artificial dataset. Various data entry errors that might have not occurred historically but have a small but non-zero probability of occurring in the future would be neglected from our study. In certain models, the probability of these orders should not be zero.

To address this limitation, we use an alternative statistical model to the MLE that assigns certain unseen dose orders a non-zero probability. This statistical model uses Good-Turing frequency estimation (GTE) to generate the probability of set of seen and unseen dose orders.

In this particular use of the GTE, the historical dataset provides the set, D_{observed}, that contains all the observed dose orders and we can compute the frequency each dose is prescribed. We defined the frequency of dose frequency, Nᵣ, as the number of doses in D_{observed} ordered with the frequency r. For example, N₁ is the number of doses that were ordered only once in the dataset. The total number of orders can be computed from the frequency of frequency data by the relation

N=Σᵣ₌₁^{∞} r Nᵣ.

The MLE estimate of the probability a specific dose is ordered r times is pᵣ=r/N and the probability of a dose not in D_{observed} is p₀=0. The GTE of the total probability of all unseen doses is estimated by

$${p}_{0} = \frac{{N}_{1}}{N} .$$

Note that this is the probability that any unseen dose will appear in the future, not the probability of a single, specific unseen dose. The probability of individual doses in D_{observed} that appear with frequency r is estimated by

$${p}_{r} = \frac{( r + 1 ) S ( {N}_{r + 1} )}{NS ( {N}_{r} )}$$

where S(Nᵣ) denotes a smoothed frequency estimate generated by linear regression.

The GTE estimates the total probability of all unseen dose orders but we needed to generate specific dose values that are unseen. Since the set of possible unseen orders is uncountably infinite, we restricted it to a finite set of more probable unseen doses, Dᵤₙₛₑₑₙ, containing N₀ elements. For each dose in Dᵤₙₛₑₑₙ, we assign an equal probability

$${\tilde{p}}_{0} = \frac{{p}_{0}}{{N}_{0}} = \frac{{N}_{1}}{{N}_{0} N} .$$

The set of all possible doses from the artificial data generator is the union of the two sets, D=D_{observed} ∪ Dᵤₙₛₑₑₙ.

To generate artificial data from the GTE of the probabilities, we computed the discrete cumulative distribution function (CDF) for the dose set D: for dose d ∈ D

CDF(d)=Σ_{d}_{ⱼ}_{<d} p(dⱼ)

where d₀<d₁< . . . <dⱼ<d_{M} are the sorted doses in D, M is the total number of possible dose orders, and p(dⱼ) is the GTE of the probability of dose dⱼ. Since the CDF is a monotonically increasing function and 0≤CDF(d)≤1, we can use the CDF to map a uniform random variable, 0≤X≤1, to a specific dose through the relation

$$d = {max}_{{d}_{j} ∈ D} CDF ( {d}_{j} ) \leq X$$

This relation was used to generate the artificial data by mapping a set of values from a uniform random distribution to a set of dose orders. Python (Python Software Foundation, version 3.5.1, Delaware, USA, 2015) was used to generate the historical frequency of frequency data, implement the GTE, and to generate the artificial dose data, the respective histograms, and to implement the following model and optimization algorithm. One hundred thousand order instances were generated for each medication.

Development of the Dosing Rule Model

Based upon our goal of decreasing alert burden to improve alert salience, we created a model to optimize CDS dosing eRules with the express intent of reducing alerts. The model evaluates a discrete number of possible rule range intervals, based upon the number of alerts each range would produce when the artificial dataset is applied (i.e., it assesses the number of theoretical alerts that would be generated with the model dosing limits). The multiple discrete combinations of dosing rule intervals considered were based on the values of where the majority of medication dose orders occur. For example, since most dosing orders for Acetaminophen occurred between zero and twenty mg/kg, multiple combinations of rule intervals between zero and twenty were considered. To find the optimal rule we assigned a score to each one being considered. We developed a measure of the quality of any given rule. Since it is mathematically easier to study the inverse of the quality of the dosing rules, we proposed this measure as the inferiority score of a dosing rule. Our inferiority measure assigns a score to each rule based on the number of theoretical alerts produced. We assume that our quality measure of a given dosing rule depends on two factors:

(1) The first factor represents the number of alerts produced by a set of simulated dosing data. Here, our goal was to empirically create dosing rules that are both clinically accurate and decrease alert burden. A dosing rule that generates a large number of alerts will have a poor quality measure.

(2) The second factor represents the length of the rule interval. This term penalizes the quality measure if the dosing interval size is larger than the length of the actual eRule, which is included to prevent the degenerative case where an arbitrary large interval that produces no alerts is acceptable.

The function returns an inferiority score for each dosing rule. A high inferiority score for a dosing rule range means that this rule is producing high amounts of alerts or that the rule interval is too large. A low inferiority score means that a dosing rule has few alerts and an acceptable interval size. Given a rule interval R=(x, y) where x is the lower dosing limit and y is the upper dosing limit and a series of dose orders, E, the measure of the inferiority, I, of a rule is defined as

$$I ( R , E ) = {w}_{1} \frac{A}{N} + {w}_{2} \frac{\vert y - x \vert}{{L}_{0}} ,$$

where A is the number of alerts generated from the set of orders, E, N is the total number of orders in the data set, L₀ is the length of the actual dosing rule interval (i.e., yₒᵣ−xₒᵣ), and w₁ and w₂, are arbitrary weights. Each term is scaled so that the alert term is less than one and the other term has a value close to unity. The weights are arbitrary and affect the relative importance between the two terms. For example, having w₁>w₂ implies that reducing the number of alerts is more important than reducing the interval length when improving the quality of the rule. Since multiple discrete combinations of dosing rules are considered, multiple inferiority scores are returned. The goal was to find the lowest inferiority score, which will be associated with a dosing rule interval.

Implementation of the Dosing Interval Optimization Algorithm

An algorithm was implemented to find the lowest inferiority score. The algorithm computed the inferiority scores for each dosing interval and stored the values in a grid where the position in the grid corresponds to the lower and upper dosing rule limit. For optimization the algorithm found the lowest score in the grid and returned the associated lower and upper rule limits of the dosing interval. Surface plots of the resulting grids were visualized using MatLab. (The MathWorks, Inc., Release 2012b, Natick, Mass., US, 2012)

Applying a Weights Simulation Algorithm

In lieu of choosing the weights a priori, we developed an algorithm that assesses the impact of various weights on the model output. We prioritized reducing the number of alerts so we chose to restrict the weights where w₁>w₂. Since it is the relative sizes of the weights to each other, we set w₂=1 and scanned the results for various values of w₁. We used a modified form of the bisection method to search through the range of weight values, w₁, looking for the critical weight values where changes in the model's dosing rule output occur. Once each critical weight value was found, the associated dosing rules returned were recorded and considered as part of the model's output. This provided a complete set of optimal dosing rules based on the relative importance of reducing the number of alerts to the interval length.

Assessing the Performance of the Model-Generated Dosing Rules

Finally, we evaluated the theoretical performance of the model-generated dosing rules compared to the actual dosing rules currently applied in the EHR. We computed the alert rates using our artificial dataset on each of the model dosing rules returned and the actual eRule. The alert rate is the percent of dosing orders that generate an alert for a given rule. We then used the alert rates to calculate the percent improvement of the returned dosing rules from the alert rate of the actual eRule as well as the number of alerts saved per one hundred thousand dosing orders.

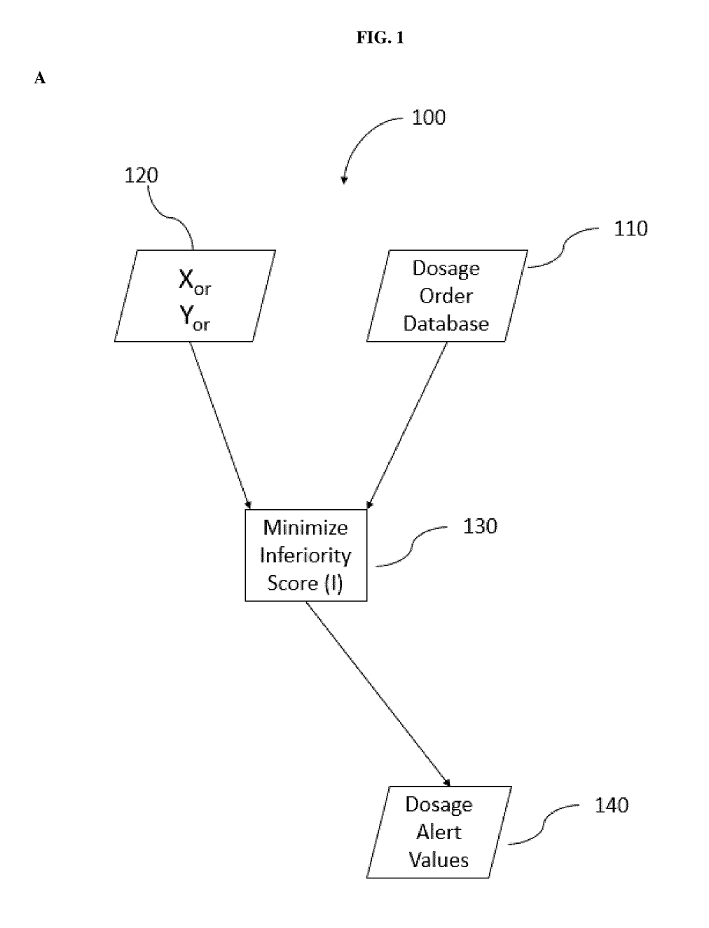

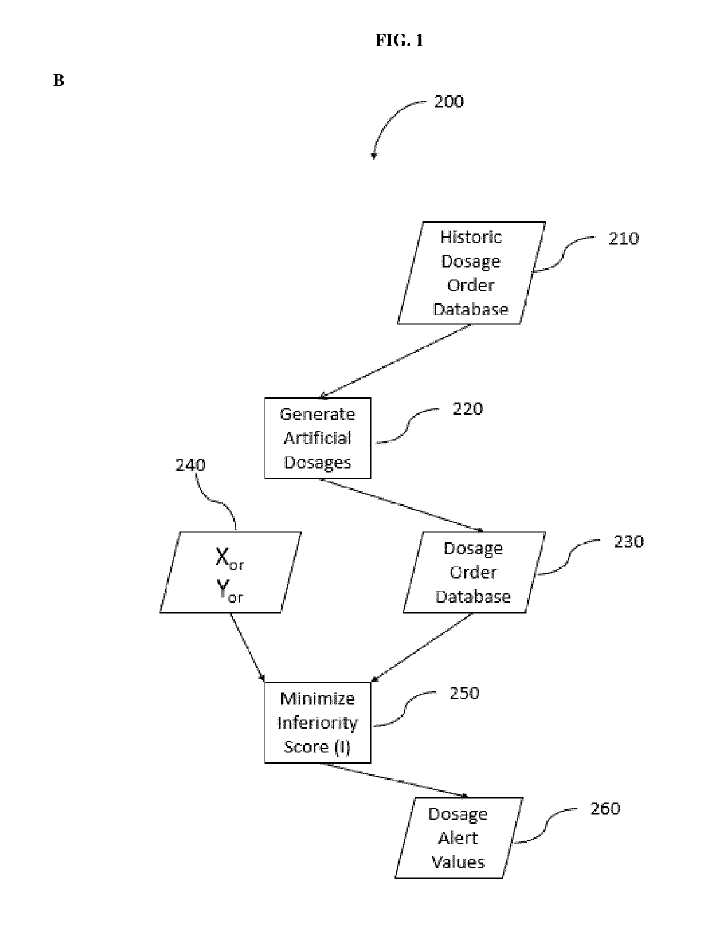

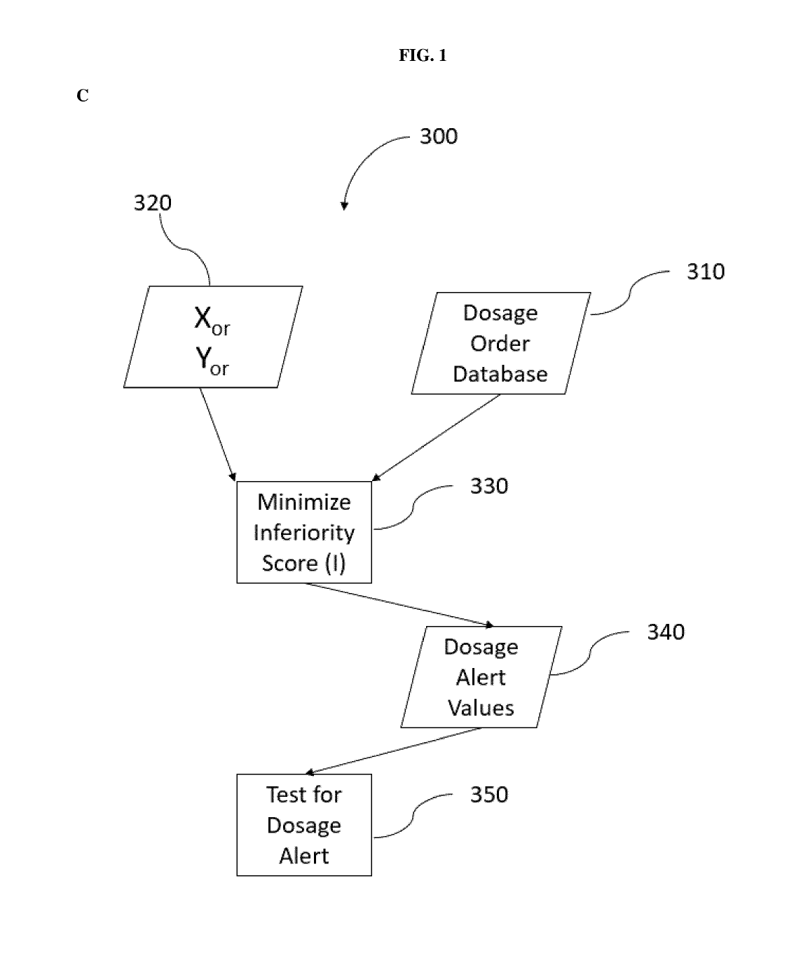

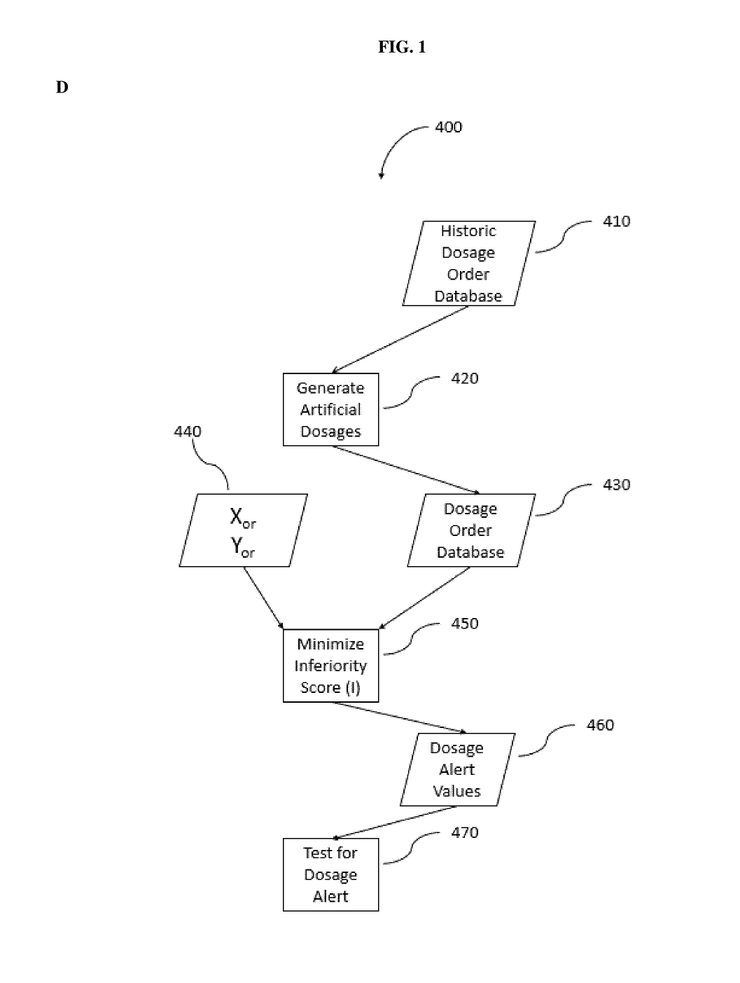

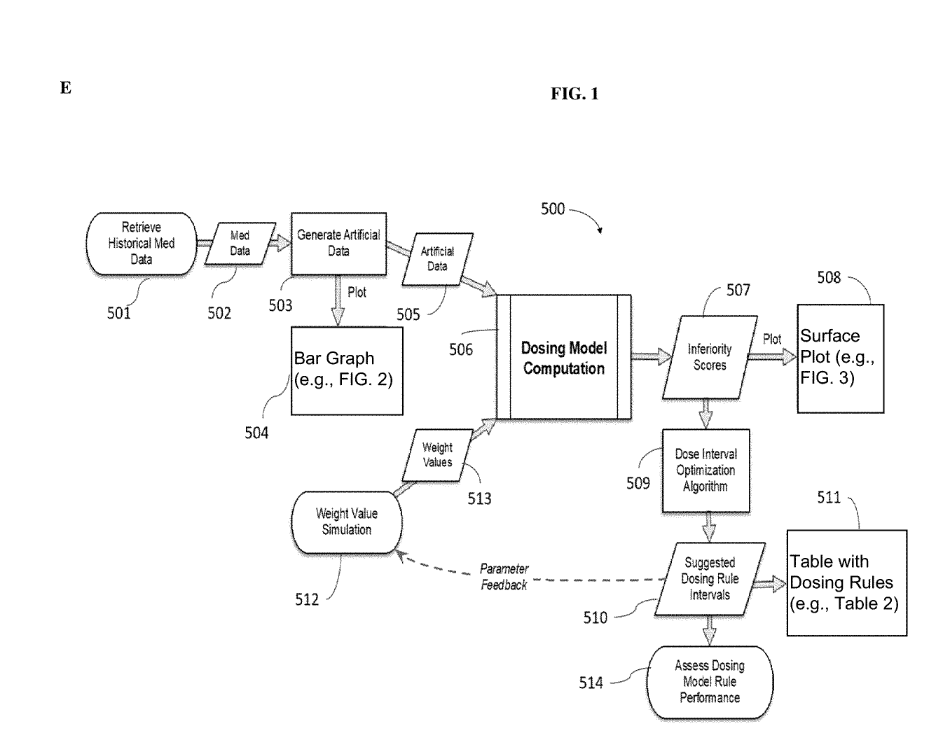

The integration and flow of certain aspects of these processes is shown in FIG. 1.

### Results

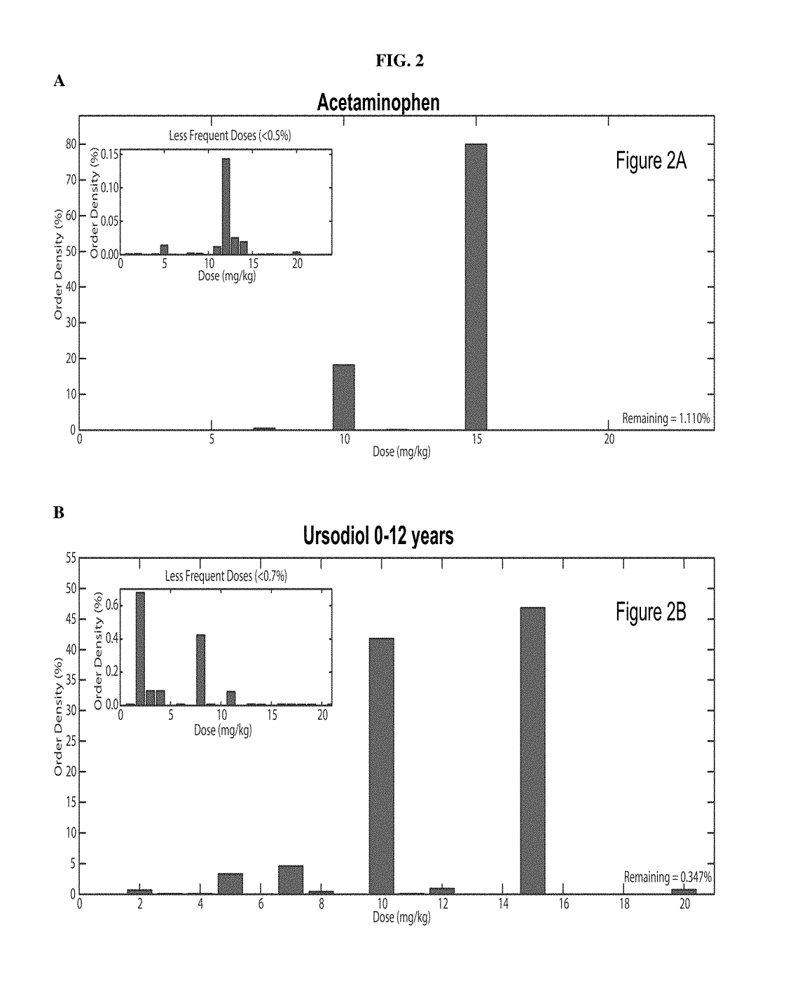

Artificial datasets were visualized using histograms (FIG. 2). The use of such insets can allow the user to better understand the distribution of the lesser frequent doses.

After the model computation used the artificial data and a set of weight values to generate inferiority scores, the optimization algorithm found the minimum value for this score and the output was visualized as shown in the example medications (FIG. 3). This visualization can permit the capability to understand how prospective changes in the dosing rule parameters would affect the score. In the case of acetaminophen, the optimal dosing range is 10-15 mg/kg. Decreasing the upper limit (to 13 mg/kg, for example) would drastically impact the inferiority score while increasing the lower limit to 13 mg/kg would not have such a profound affect.

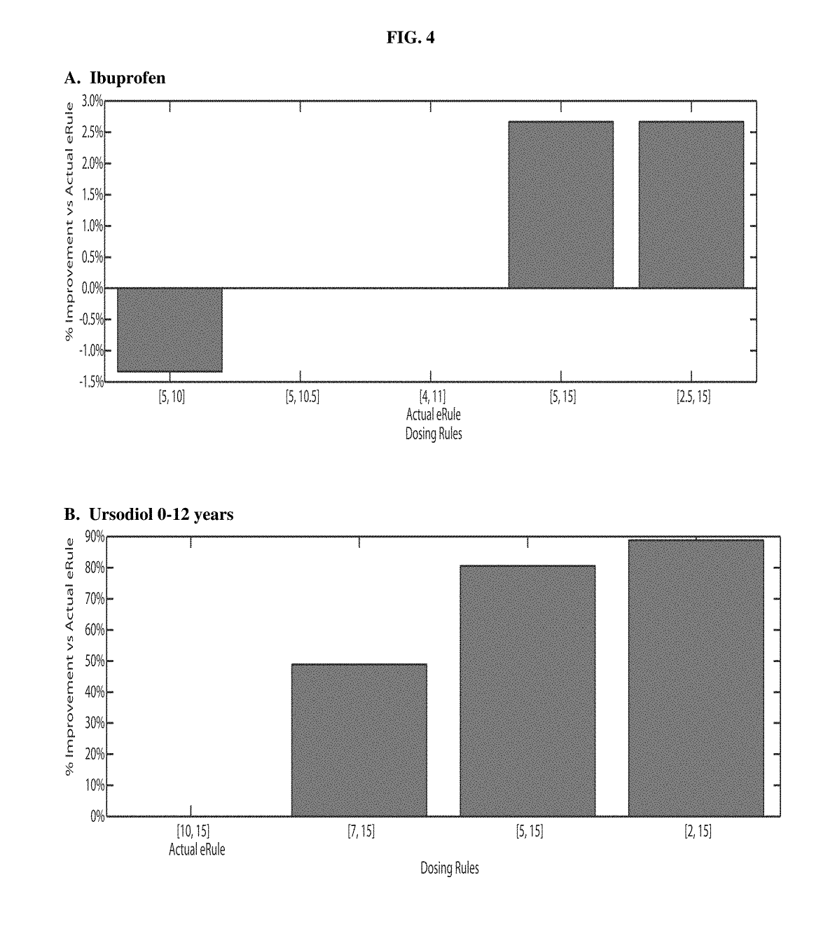

Table 2 represents the results of the model output and weight simulation experiments; the different rules are the result of the model computation with the weights selected by the weight simulation algorithm. Varying the weights on the model terms output differing optimal rule ranges, with varying affects on alert rates and alerts saved. These tables can allow a user to quickly scan the model output and select most clinically-appropriate rule thresholds that have the largest reductions in alert burden. FIG. 4 demonstrates these gains graphically.

TABLE 2
Weight Value Simulation Dosing Rule Results

| Rule Range | | | | Alerts Saved per |
|---|---|---|---|---|
| mg/kg | | | % improvement | 100K Orders |
| Lower | Upper | Alert Rate | vs Actual eRule | vs Actual eRule |

A

| | | | | | |
|---|---|---|---|---|---|
| Acetaminophen | | | | | |
| Actual eRule | 5 | 15 | 1.20 | Not applicable | Not applicable |
| Model Rule #1 | 10 | 15 | 1.73 | −44.19% | −529 |
| Model Rule #2 | 7.5 | 15 | 1.21 | −1.00% | −12 |
| Model Rule #3 | 5 | 15 | 1.20 | 0.00% | 0 |
| Model Rule #4 | 2.5 | 15 | 1.19 | 0.25% | 3 |
| Model Rule #5 | 1 | 15 | 1.19 | 0.33% | 4 |
| Model Rule #6 | 2.5 | 20 | 1.19 | 0.50% | 6 |
| Model Rule #7 | 1 | 20 | 1.19 | 0.58% | 7 |

B

| | | | | | |
|---|---|---|---|---|---|
| Ibuprofen | | | | | |
| Actual eRule | 4 | 11 | 0.75 | Not Applicable | Not Applicable |
| Model Rule #1 | 5 | 10 | 0.76 | −0.80% | −6 |
| Model Rule #2 | 5 | 10.5 | 0.75 | −0.27% | −2 |
| Model Rule #3 | 5 | 15 | 0.73 | 2.40% | 18 |
| Model Rule #4 | 2.5 | 15 | 0.73 | 2.66% | 20 |

C

| | | | | | |
|---|---|---|---|---|---|
| Diphenhydramine | | | | | |
| Actual eRule | 0.1 | 1.5 | 1.28 | Not Applicable | Not Applicable |
| Model Rule #1 | 0.5 | 1 | 3.41 | −166.64% | −2133 |
| Model Rule #2 | 0.5 | 1.25 | 1.36 | −6.25% | −80 |
| Model Rule #3 | 0.25 | 1.25 | 1.28 | −0.16% | −2 |
| Model Rule #4 | 0.25 | 1.5 | 1.28 | 0.00% | 0 |
| Model Rule #5 | 0 | 1.5 | 1.28 | 0.08% | 1 |
| Model Rule #6 | 0 | 5 | 1.26 | 1.64% | 21 |

D

| | | | | | |
|---|---|---|---|---|---|
| Amoxicillin | | | | | |
| Actual eRule | 8 | 45 | 2.06 | Not Applicable | Not Applicable |
| Model Rule #1 | 40 | 45 | 13.27 | −544.03% | −11207 |
| Model Rule #2 | 25 | 45 | 7.03 | −241.41% | −4973 |
| Model Rule #3 | 20 | 45 | 5.26 | −155.53% | −3204 |
| Model Rule #4 | 12.5 | 45 | 2.33 | −13.25% | −273 |
| Model Rule #5 | 12.5 | 50 | 1.70 | 17.52% | 361 |
| Model Rule #6 | 10 | 50 | 1.43 | 30.58% | 630 |
| Model Rule #7 | 7.5 | 50 | 1.42 | 31.07% | 640 |
| Model Rule #8 | 4.5 | 50 | 1.41 | 31.36% | 646 |

E

| | | | | | |
|---|---|---|---|---|---|
| Ursodiol 0-12 | | | | | |
| Actual eRule | 10 | 15 | 10.25 | Not Applicable | Not Applicable |
| Model Rule #1 | 10 | 15 | 10.25 | 0.00% | 0 |
| Model Rule #2 | 7 | 15 | 5.18 | 49.43% | 5068 |
| Model Rule #3 | 5 | 15 | 1.98 | 80.68% | 8271 |
| Model Rule #4 | 2 | 15 | 1.16 | 88.73% | 9097 |

F

| | | | | | |
|---|---|---|---|---|---|
| Ursodiol 12-99 | | | | | |
| Actual eRule | 2 | 5 | 50.62 | Not Applicable | Not Applicable |
| Model Rule #1 | 5 | 10 | 16.08 | 68.24% | 34538 |
| Model Rule #2 | 4 | 10 | 11.05 | 78.17% | 39564 |
| Model Rule #3 | 4 | 14.5 | 4.08 | 91.94% | 46535 |
| Model Rule #4 | 4 | 15 | 3.56 | 92.96% | 47052 |

Discussion

Current methods for adjusting medication dosing rules have many challenges and are largely predicated on time-consuming procedures that are dependent on expert opinion and knowledge. The approach outlined in this study represents an alternative set of procedures for setting and adjusting electronic rule (eRule) dosing parameters, one that is empiric, scalable, and scientific. It uses data generated by the EHR through previous activities by prescribers (empiric), is automatable (thus scalable and addressing the current state of entirely manual adjustment) and can serve as a basis for a CDS learning system. This secondary use of data means that resources used to input data into the model are minimal—there is no requirement for gathering primary data as substrate. The mathematical model and algorithm are a feasible alternative solution to adjusting medication dosing rules.

Their commonality of some of the candidate medications used in this study allows for plentiful data to serve as the foundation for our test data. They can represent one end of the prescribing spectrum. These medications are also commonly used for a multitude of clinical indications and the eRules for these drugs have been heavily customized with great scrutiny. The results (Table 2) reflect as such, in that the dosing intervals are generally very liberal, which limits our ability to demonstrate a large alert savings. Heavy customization of the eRules has also led to a pre-study adjustment that limits the alert savings results, especially with Acetaminophen, Ibuprofen, and Diphenhydramine, all of which are dosed heterogeneously in our clinical environment.

One real value of the model and algorithm will be demonstrated in less mainstream circumstances; in situations where dosing is more heterogeneous, rule customization has not been performed, or customization thresholds have been configured that do not match clinical practice. As such, the comparison of the model-generated dosing rules to the customized eRules for Amoxicillin and Ursodiol demonstrate a greater alert savings.

As with all dosage alerts systems, automating the process of dosing rule adjustment through this method does not alleviate the need for clinical judgment. Mathematical modeling using historic prescribing data can generate more clinically-appropriate electronic dosing rule parameters. This approach represents a scalable solution that could decrease alert fatigue and decrease medication dosing errors.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The techniques described herein can be implemented on any suitable platform, including but not limited to digital electronic circuitry, computer hardware, firmware, software, or any combinations thereof. The techniques can be implemented as a computer program product ((e.g., a computer program tangibly embodied in an information carrier, such as in a machine usable or machine-readable storage device) (e.g., a magnetic or digital medium such as a Universal Serial Bus (USB) storage device, a tape, hard disk drive, compact disk, digital video disk (DVD), etc.)) or in a propagated signal, for execution by, or to control the operation of, data processing apparatus (e.g., a programmable processor, a computer, or multiple computers). A computer program, such as the computer program(s) described above, can be written in any form using any suitable programming language, including compiled or interpreted languages, and can be deployed in any form, including as a stand-alone program or as a module, component, subroutine, or other unit suitable for use in a computing environment. A computer program that might implement the techniques discussed herein can be deployed to be executed on one computer or on multiple computers at one site or distributed across multiple sites and interconnected by a communication network. Method steps can be performed by one or more programmable processors executing a computer program to perform functions by operating on input data and generating output. The one or more programmable processors can execute instructions in parallel, and/or can be arranged in a distributed configuration for distributed processing. Method steps also can be performed by, and an apparatus can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. In certain embodiments, a processor will receive instructions and data from a read only memory or a random-access memory or both. Elements of a computer can include at least one processor for executing instructions and one or more memory devices for storing instructions and data. In other embodiments], a computer also can include, or be operatively coupled to receive data from or transfer data to, or both, one or more mass storage devices for storing data (e.g., magnetic, magneto optical disks, or optical disks). Information carriers suitable for embodying computer program instructions and data include all forms of non-volatile memory, including by way of example semiconductor memory devices (e.g., EPROM, EEPROM, and flash memory devices); magnetic disks (e.g., internal hard disks or removable disks); magneto optical disks; and CD ROM and DVD-ROM disks. The processor and the memory can be supplemented by, or incorporated in, special purpose logic circuitry.

To provide for interaction with a user, the methods or techniques disclosed herein can be implemented on a computer having a display device, e.g., a cathode ray tube (CRT) or liquid crystal display (LCD) monitor, for displaying information to the user and a keyboard and a pointing device (e.g., a mouse or a trackball), by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The methods or techniques disclosed herein can be implemented in a computing system that includes a back-end component (e.g., as a data server), or that includes a middleware component (e.g., an application server), or that includes a front-end component (e.g., a client computer having a graphical user interface or a Web browser through which a user can interact with an implementation), or any combination of such back end, middleware, or front end components. Components can be interconnected by any form or medium of digital data communication, e.g., a communication network. Examples of communication networks include a local area network (LAN) and a wide area network (WAN) (e.g., the internet).

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications, substitutions, changes and equivalents of the invention in addition to those described herein will become apparent to those skilled in the art from the foregoing description and the accompanying figures. Such modifications, substitutions, changes and equivalents are intended to fall within the scope of the appended claims.

What is claimed is:

1. A computer implemented method for generating dosage alert values xₘᵢₙ and yₘᵢₙ for a drug, the method comprising
minimizing I with respect to x, y, w₁, and w₂ for a dosage order database; wherein I=w₁*(A/N)+w₂*(|y−x|/L₀),
the dosage order database comprises dosage orders for the drug,
a dosage in the dosage order database that is less than x generates an alert,
a dosage in the dosage order database that is greater than y generates an alert,
A is the number of alerts generated by the dosage orders in the dosage order database with given values of x and y,
N is the total number of orders in the dosage order database for the drug,
w₁ and w₂ are weights,
L₀=yₒᵣ−xₒᵣ,
xₒᵣ is an original dosage alert value for the drug where a dosage in the dosage order database below xₒᵣ will generate an alert,
yₒᵣ is an original dosage alert value for the drug where a dosage in the dosage order database above yₒᵣ will generate an alert,
xₘᵢₙ is the value of x when I is minimized, and
yₘᵢₙ is the value of y when I is minimized.

2. The method of claim 1, wherein the drug is abatacept, abiraterone, acetaminophen, acetaminophen/hydrocodone, adalimumab, adderall, albuterol sulfate, alprazolam, amitriptyline, amlodipine, amoxicillin, amoxicillin-pot clavulanate, amphetamine mixed salts, analgesics-narcotic, analgesics-nonnarcotic, antianxiety agents, antiasthmatic, anticonvulsant, antidepressants, antihistamines, antiinfectives, anti-rheumatic, aripiprazole, atazanavir, ativan, atorvastatin, azithromycin, bevacizumab, budesonide, budesonide/formoterol, buprenorphine, capecitabine, celecoxib, cephalosporins, chlorothiazide, ciclosporin ophthalmic emulsion, cinacalcet, ciprofloxacin, citalopram, clindamycin, clindamycin HCl, clindamycin palmitate HCl, clonazepam, codeine, corticosteroids, cyclobenzaprine, cymbalta, dabigatran, darbepoetin alfa, darunavir, denosumab, dexlansoprazole, diazepam, diphenhydramine HCl, doxycycline, duloxetine, elvitegravir/cobicistat/emtricitabine/tenofovir, emtricitabine/rilpivirine/tenofovir disoproxil fumarate, emtricitabine/tenofovir/efavirenz, enoxaparin, epoetin alfa, esomeprazole, eszopiclone, etanercept, everolimus, ezetimibe, ezetimibe/simvastatin, fenofibrate, filgrastim, fingolimod, fluticasone propionate, fluticasone propionate/salmeterol, fluticasone/salmeterol, furosemide, gabapentin, gastrointestinal agents, glatiramer, hydrochlorothiazide, hydromorphone HCl, hypnotics, ibuprofen, imatinib, infliximab, insulin aspart, insulin detemir, insulin glargine, insulin lispro, interferon beta 1b, ipratropium bromide/salbutamol, laxatives, ledipasvir/sofosbuvir, lenalidomide, levetiracetam, levothyroxine, lexapro, lidocaine, liraglutide, lisdexamfetamine, lisinopril, local anesthetics-parenteral, loratadine, lorazepam, losartan, lyrica, melatonin, meloxicam, memantine, metformin, methadone HCl, methylphenidate, metoprolol, metoprolol, mometasone, naproxen, olmesartan, olmesartan/hydrochlorothiazide, omalizumab, omega-3 fatty acid ethyl esters, omeprazole, ondansetron HCl, ophthalmic, oxycodone, palivizumab, pantoprazole, pemetrexed, penicillin v potassium, penicillins, pneumococcal conjugate vaccine, polymyxin b-trimethoprim, prednisolone sodium phosphate, prednisone, pregabalin, quetiapine, rabeprazole, raloxifene, raltegravir, ranibizumab, ranitidine HCl, rituximab, rivaroxaban, rocuronium bromide, rosuvastatin, salbutamol, sevelamer, sildenafil, sitagliptin, sitagliptin/metformin, sofosbuvir, solifenacin, stimulants, sulfamethoxazole-trimethoprim, tacrolimus, tadalafil, telaprevir, tenofovir/emtricitabine, testosterone gel, tiotropium bromide, tramadol, trastuzumab, trazodone, ulcer drugs, ursodiol, ursodiol, ustekinumab, valproate, valsartan, viagra, wellbutrin, xanax, zoloft, or zostavax.

3. The method of claim 1, wherein the drug is acetaminophen, albuterol sulfate, amoxicillin, amoxicillin-pot clavulanate, chlorothiazide, clindamycin HCl, clindamycin palmitate HCl, diazepam, diphenhydramine HCl, epoetin alfa, furosemide, hydromorphone HCl, levetiracetam, lorazepam, melatonin, methadone HCl, ondansetron HCl, penicillin v potassium, polymyxin b-trimethoprim, prednisolone sodium phosphate, ranitidine HCl, rocuronium bromide, sulfamethoxazole-trimethoprim, tacrolimus, or ursodiol.

4. The method of claim 1, wherein the drug is acetaminophen, amoxicillin, diphenhydramine, ibuprofen, ursodiol for ages 0-12, or ursodiol for ages 12-99.

5. The method of claim 1, wherein the drug is acetaminophen, amoxicillin, diphenhydramine, ibuprofen, or ursodiol for ages 0-12.

6. The method of claim 1, wherein the dosage order database is a historic dosage order database.

7. The method of claim 1, wherein the dosage order database is generated by adding additional dosage data to a historic dosage order database.

8. The method of claim 1, wherein the dosage order database is generated by adding additional dosage data to a historic dosage order database and the additional dosage data is generated comprising using a numerical model applied to the historic dosage order database, where the numerical model comprises a frequentist statistical model or a Baysian model.

9. The method of claim 1, wherein the dosage order database is generated by adding additional dosage data to a historic dosage order database and the additional dosage data is generated comprises using a numerical model applied to the historic dosage order database, where the numerical model comprises a Good-Turing frequency estimation.

10. The method of claim 1, wherein w₁ is set to be greater than w₂.

11. The method of claim 1, wherein w₂ is set to be equal to one.

12. The method of claim 1, wherein w₁ is set to be greater than w₂ and w₂ is set to be equal to one.

13. The method of claim 1, wherein the dosage order database comprises at least about 10,000 dosage orders, at least about 100,000 dosage orders, at least about 500,000 dosage orders, or at least about 2,000,000 dosage orders.

14. The method of claim 1, wherein the xₘᵢₙ and yₘᵢₙ resulting from the minimization provide a percent alert rate improvement which is at least about 0%, at least about 0.1%, at least about 0.2%, at least about 0.3%, at least about 0.4%, at least about 0.5%, at least about 1%, at least about 1.5%, at least about 10%, at least about 25%, at least about 50%, at least about 75%, at least about 80%, at least about 85%, at least about 90%, at least about 95%, or at least about 99%.

15. The method of claim 1, wherein the xₘᵢₙ and yₘᵢₙ resulting from the minimization provide a number of dosage alerts saved per one hundred thousand dosage orders which is at least about 0, at least about 10, at least about 50, at least about 100, at least about 250, at least about 500, at least about 1000, at least about 5000, at least about 10,000, at least about 20,000, at least about 30,000, at least about 40,000, at least about 50,000, or at least about 90,000.

16. A non-transitory computer-readable medium comprising computer program instructions executable by a computer processor to execute a method for generating dosage alert values xₘᵢₙ and yₘᵢₙ for a drug according to the method of claim 1.

17. The non-transitory computer-readable medium of claim 16, wherein the drug is acetaminophen, amoxicillin, diphenhydramine, ibuprofen, or ursodiol for ages 0-12.

18. The non-transitory computer-readable medium of claim 16, wherein the xₘᵢₙ and yₘᵢₙ resulting from the minimization provide a percent alert rate improvement which is at least about 0%, at least about 0.1%, at least about 0.2%, at least about 0.3%, at least about 0.4%, at least about 0.5%, at least about 1%, at least about 1.5%, at least about 10%, at least about 25%, at least about 50%, at least about 75%, at least about 80%, at least about 85%, at least about 90%, at least about 95%, or at least about 99%.

19. The non-transitory computer-readable medium of claim 16, wherein the xₘᵢₙ and yₘᵢₙ resulting from the minimization provide a number of dosage alerts saved per one hundred thousand dosage orders which is at least about 0, at least about 10, at least about 50, at least about 100, at least about 250, at least about 500, at least about 1000, at least about 5000, at least about 10,000, at least about 20,000, at least about 30,000, at least about 40,000, at least about 50,000, or at least about 90,000.

20. A computer implemented method for generating dosage alerts for a drug, the method comprising
inputting a dosage request for a drug, and
generating a dosage alert if (a) the dosage request for the drug is less than xₘᵢₙ, (b) the dosage request for the drug is greater than yₘᵢₙ, or (c) both;
wherein xₘᵢₙ and yₘᵢₙ for the drug are generated using the method of claim 1.

21. The method of claim 20, wherein the inputting occurs using a keyboard, a pointing device, a microphone, a touch screen, or a combination thereof.

22. The method of claim 20, wherein the dosage request is performed by a prescriber or a person who assists a prescriber.

23. The method of claim 20, wherein the dosage alert is a sound, a light, a text message, a message on a screen, a spoken message, a tactile sensation, or a combination thereof.

24. The method of claim 20, wherein the dosage alert is received by a prescriber or a person who assists a prescriber.

25. The method of claim 20, wherein the drug is acetaminophen, amoxicillin, diphenhydramine, ibuprofen, or ursodiol for ages 0-12.

26. The method of claim 20, wherein the xₘᵢₙ and yₘᵢₙ resulting from the minimization provide a percent alert rate improvement which is at least about 0%, at least about 0.1%, at least about 0.2%, at least about 0.3%, at least about 0.4%, at least about 0.5%, at least about 1%, at least about 1.5%, at least about 10%, at least about 25%, at least about 50%, at least about 75%, at least about 80%, at least about 85%, at least about 90%, at least about 95%, or at least about 99%.

27. The method of claim 20, wherein the xₘᵢₙ and yₘᵢₙ resulting from the minimization provide a number of dosage alerts saved per one hundred thousand dosage orders which is at least about 0, at least about 10, at least about 50, at least about 100, at least about 250, at least about 500, at least about 1000, at least about 5000, at least about 10,000, at least about 20,000, at least about 30,000, at least about 40,000, at least about 50,000, or at least about 90,000.

28. A non-transitory computer-readable medium comprising computer program instructions executable by a computer processor to execute a method for generating dosage alerts for a drug according to the method of claim 20.

29. The non-transitory computer-readable medium of claim 28, wherein the inputting occurs using a keyboard, a pointing device, a microphone, a touch screen, or a combination thereof.

30. The non-transitory computer-readable medium of claim 28, wherein the dosage request is performed by a prescriber or a person who assists a prescriber.

31. The non-transitory computer-readable medium of claim 28, wherein the dosage alert is a sound, a light, a text message, a message on a screen, a spoken message, a tactile sensation, or a combination thereof.

32. The non-transitory computer-readable medium of claim 28, wherein the dosage alert is received by a prescriber or a person who assists a prescriber.

33. The non-transitory computer-readable medium of claim 28, wherein the drug is acetaminophen, amoxicillin, diphenhydramine, ibuprofen, or ursodiol for ages 0-12.

34. The non-transitory computer-readable medium of claim 28, wherein the xₘᵢₙ and yₘᵢₙ resulting from the minimization provide a percent alert rate improvement which is at least about 0%, at least about 0.1%, at least about 0.2%, at least about 0.3%, at least about 0.4%, at least about 0.5%, at least about 1%, at least about 1.5%, at least about 10%, at least about 25%, at least about 50%, at least about 75%, at least about 80%, at least about 85%, at least about 90%, at least about 95%, or at least about 99%.

35. The non-transitory computer-readable medium of claim 28, wherein the xₘᵢₙ and yₘᵢₙ resulting from the minimization provide a number of dosage alerts saved per one hundred thousand dosage orders which is at least about 0, at least about 10, at least about 50, at least about 100, at least about 250, at least about 500, at least about 1000, at least about 5000, at least about 10,000, at least about 20,000, at least about 30,000, at least about 40,000, at least about 50,000, or at least about 90,000.

36. Dosage alert values xₘᵢₙ and yₘᵢₙ for the drug, as determined by the method of claim 1.
